(19) United States
(12) Patent Application Publication
(10) Pub. No.: US 2005/0209577 A1
(43) Pub. Date: Sep. 22, 2005

(54) Female disposable leakproof sanitary pantie

(76) Inventor: Hung Wai Tong, Hong Kong (CN)

Correspondence Address: Hung Wai Tong;FLAT 'D'. 10/F., BLK. 2, ESTORIL COURT, 55 GARDEN RD., MIDLEVEL HONG KONG., HONG KONG, CN

(21) Appl. No.: 10/801,923

(22) Filed: Mar. 17, 2004

(51) Int. Cl. A61F 13/15; A61F 13/20

(52) U.S. Cl. 604/387; 604/358

(57) ABSTRACT

This invention provides a female disposable leakproof sanitary pantie including the absorbent material is being sewn onto the two curve edges inside the pantie to prevent fluid leakage. In addition, there are four straps Or four Velcro on the both sides of pantie for easy removal instead of through the legs thus prevent soiling while changing. Preferably the pantie is made of non-woven fabric but it may instead be made from other disposable material. It can be in different color such as white, red, black, pink, blue, etc.

### CROSS-REFERENCE TO RELATED APPLICATIONS

“Not Applicable”

### STATEMENT REGARDING FEDERALLY SPONSORED RESEARCH OR DEVELOPMENT

“Not Applicable”

### REFERENCE TO SEQUENCE LISTING, A TABLE, OR A COMPUTER PROGRAM LISTING COMPACT DISK APPENDIX

“Not Applicable”

### BACKGROUND OF THE INVENTION

Traditional pantie suffer from the disadvantage that, when it is being used during female period with sanitary napkin, problems of fluid leakage and removal of sanitary napkin inside the pantie are often exist. Moreover, it is inconvenience for female to change her fluid polluted pantie when she was out, as she has to takeoff her shoes then to takeoff the pantie through her legs. The fluid polluted pantie often soiled the clothes while changing the pantie.

### BRIEF SUMMARY OF THE INVENTION

This invention provides a pantie including the absorbent material is being sewn onto the two curve edges inside the pantie to prevent fluid leakage. In addition, there are four straps Or four Velcro on the both sides of pantie for easy removal instead of through the legs thus prevent soiling while changing.

The first object of this invention is to provide a sanitary pantie for female during period that prevents fluid leakage. The second object is to provide a sanitary pantie for female during period that keeps the sanitary napkin in place inside the pantie. The last object is to provide a sanitary pantie for female during period that prevents soiling the clothes while changing the pantie.

### BRIEF DESCRIPTION OF THE SEVERAL VIEWS OF THE DRAWING

FIG. 1 shows the outer view of the whole pantie that its removal device is straps;

FIG. 2 shows the inner view of the whole pantie that its removal device is straps;

FIG. 3 shows the outer view of the whole pantie that its removal device is Velcro;

FIG. 4 shows the inner view of the whole pantie that its removal device is Velcro.

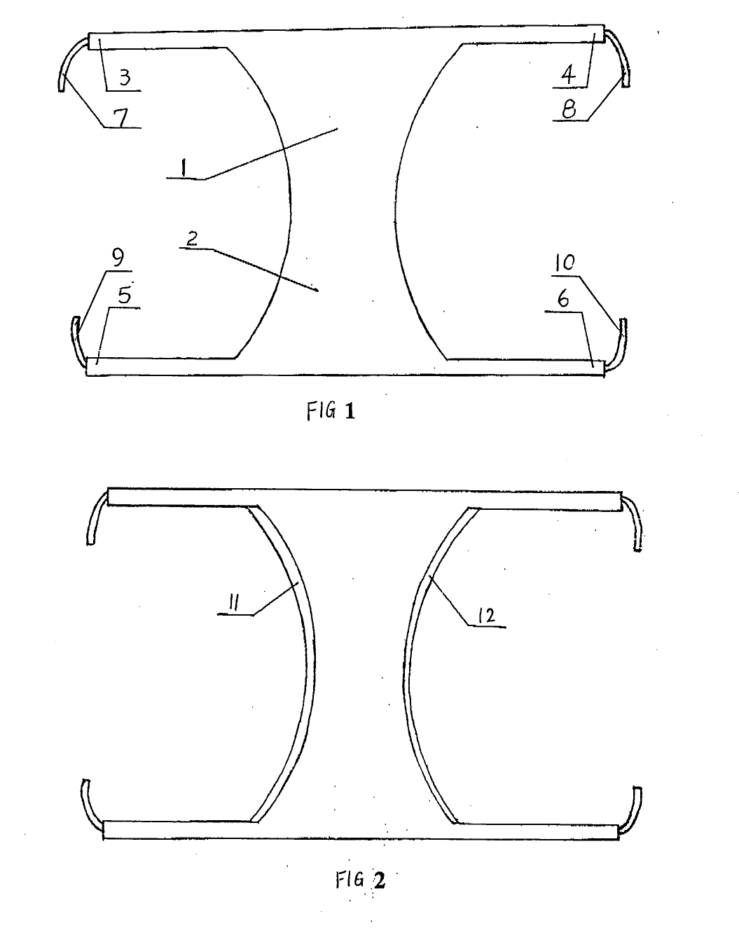

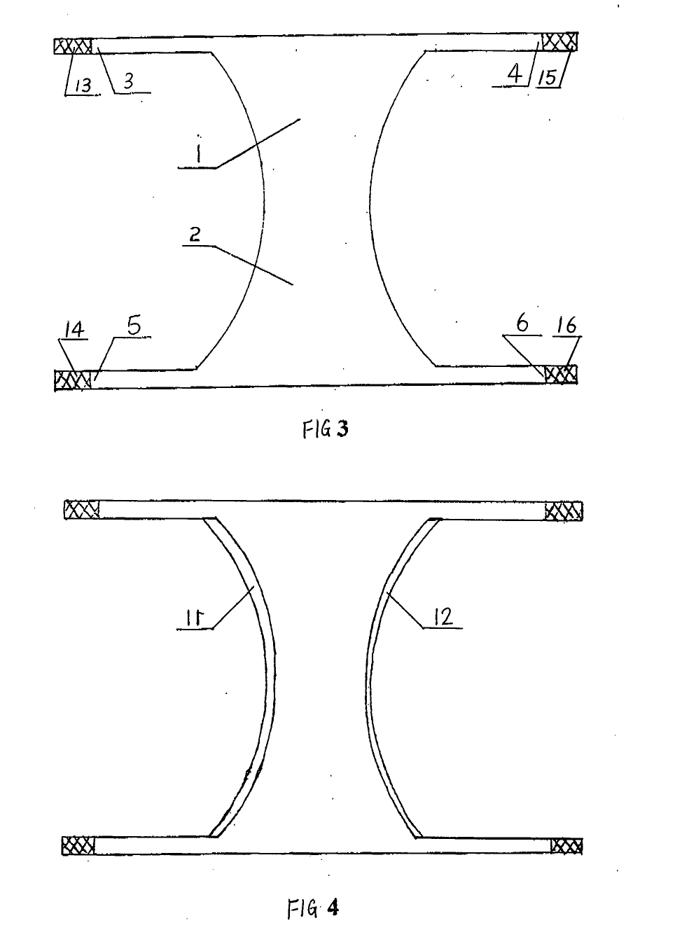

As shown in FIG. 1 and FIG. 3, the pantie is comprised of anterior part 1 and posterior part 2. There are removal device located at the waist 3, 4, 5, 6. The removal device can be straps 7, 8, 9, 10 as shown in FIG. 1 Or Velcro 13, 14, 15, 16 as shown in FIG. 3.

As shown in FIG. 2 and FIG. 4, the absorbent material is being sewn onto the two curve edges 11, 12 inside the pantie to prevent fluid leakage and removal of sanitary napkin.

### DETAILED DESCRIPTION OF THE INVENTION

To put on the pantie, simply wrap the anterior part 1, the left waist 4 and the posterior part 2, the left waist 6 towards the left waist of your body, then tight up the straps 8 and 10 Or fasten the Velcro 15 and 16. Also wrapped the right waist 3 and 5 towards the right waist of your body, then tight up the straps 7 and 9 Or fasten the Velcro 13 and 14.

To remove the pantie, simply undo all the straps Or unfasten all the Velcro.

Preferably the pantie is made of non-woven fabric but it may instead be made from other disposable material. It can be in different color such as white, red, black, pink, blue, etc.

1. I claim: the absorbent material is being sewn onto the two curve edges inside the pantie to prevent fluid leakage and removal of sanitary napkin inside the pantie for female during period.

2. I claim: there are four straps on the both sides of pantie for easy removal instead of through the legs thus prevents soiling the clothes while changing the pantie.

3. I claim: there are four Velcro on the both sides of pantie for easy removal instead of through the legs thus prevents soiling the clothes while changing the pantie.
